(19) United States
(12) Patent Application Publication
(10) Pub. No.: US 2012/0220822 A1
(43) Pub. Date: Aug. 30, 2012

(54) HAND-HELD ENDOSCOPIC DEVICE WITH A ROTATION CONTROL

(75) Inventor: YUNG-HSIU CHEN, Taipei (TW)

(73) Assignee: THREE-IN-ONE ENTERPRISES CO., LTD., Taipei (TW)

(21) Appl. No.: 13/035,558

(22) Filed: Feb. 25, 2011

(30) Foreign Application Priority Data
Feb. 25, 2011 (TW) 099105558

(51) Int. Cl. A61B 1/04

(52) U.S. Cl. 600/109

(57) ABSTRACT

The present invention is a hand-held endoscopic device with a rotation control, having a mainframe, a flexible cable, and a tube module. Moreover, the tube module includes a probe, a image capturing element and a sheath; besides, the second front end of the sheath has an opening which includes a mirror to allow the user to rotate the opening of the sheath in accordance with the situation, so that the image capturing element can reflect the light through the mirror to capture the image from different perspectives.

### FIELD OF THE INVENTION

The present invention relates to a hand-held endoscope with a rotation control; particularly, this invention relates to an endoscope that can rotate 360 degrees.

### BACKGROUND OF THE INVENTION

Now the endoscope has largely improved the performance of observing the inside of a certain thing by penetrating it and is widely applied in all fields. The old endoscopes mostly consist of a large monitor, a light source, a power source and an endoscope; and these equipments were heavier and larger at that time. After years of research and development, the size of an endoscope has been reduced to portable size, which is indeed a great improvement.

The hand-held and portable endoscope, such as what is mentioned in the invention in the patent of Taiwan, the Republic of China “endoscope with adjustable visual angle” (Patent Number: M372177). The hand-held and portable endoscope captures images through rotating the image capturing module using a flexible cable, or further let the two draglines tie or release each other through rotating the adjustment button on the cable base, so the flexible tubes would bend towards each other to adjust its visual angle; however, this method has some problems. The problems are listed as follows:
1. If the device is used in a dense examination area, due to the narrowness of the area, the flexible tube is not allowed to be bent; therefore the endoscope can not capture the surrounding images easily.
2. The device goes deep into the examination area through a flexible wire or tube. Because the cable or tube is made of certain soft material, if it is used in a highly dense area, it will be difficult for the cable or tube to reach deep into the desired position.
3. In order for the device to capture the image of a wide angle, the cable or the inside of the tube need to be processed so as to be bent, such as dragline, adjustment button, cable base, etc, which would increase the cost of manufacturing.

From above statements, how to reduce costs and develop a hard structure of an endoscope that can be used in a dense examination area is an important issue that needs to be sorted out immediately.

### SUMMARY OF THE INVENTION

In order to solve the problems above, the present invention provides a hand-held endoscope having a rotation control to make up for the shortage of the current technology and correct the flaws in prior art.

One of the characteristics of the hand-held endoscope device provided by the present invention is that the device has a probe and a sheath; in which, the sheath can be mounted on the outside of the probe and connected with the probe by means of screw thread.

Another characteristic of the hand-held endoscope device provided by the present invention is that both the probe and the sheath have a connection part, and the connection parts have a screw thread area and a smooth area, when the sheath is mounted on the onside of the probe, these two parts are connected through the screw threads. When the screw thread of the screw thread area is further screwed into the smooth area, because the two sets of the screw thread do not occlude each other anymore; meanwhile, if the sheath is further rotated, the sheath will be rotated without occluding the probe. Therefore, users can rotate the sheath without the sheath coming undone from the probe.

Yet another characteristic of the hand-held endoscope device provided by the present invention is that a second front end of the sheath has an opening, inside which a mirror is installed, so that the image capturing element can reflect the light through the mirror, so as to capture images from different perspectives.

### BRIEF DESCRIPTION OF THE DRAWINGS

FIG. 1 is the schematic diagram showing a hand-held endoscopic device of the present invention.

FIG. 2 is the cross-sectional image of the second front end of the present invention.

FIG. 3 is the schematic diagram showing the coupling means of the sheath and the probe.

FIG. 4 is another embodiment of the present invention.

FIG. 5 is yet another embodiment of the present invention.

FIG. 6 is the schematic diagram showing the first front end of the probe of the present invention.

### DETAILED DESCRIPTION OF THE PREFERRED EMBODIMENT

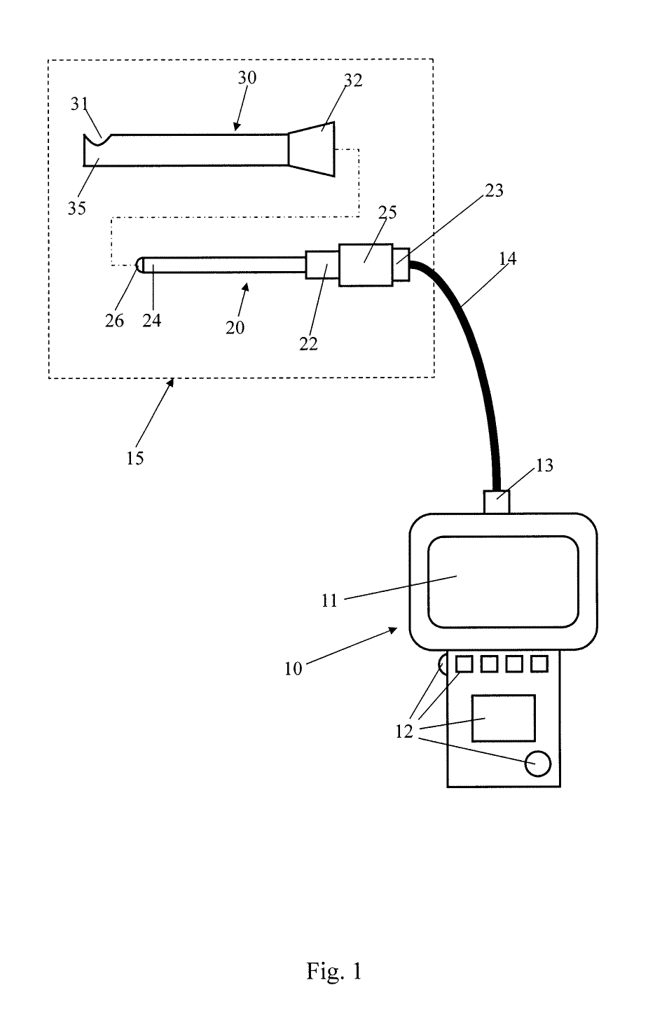

Please refer to FIG. 1, the schematic diagram of the present invention. From FIG. 1, the overall shape and structure can be understood. The present invention is a hand-held endoscopic device with a rotation control, having a hand-held mainframe 10, a flexible cable 14, and a tube module 15; moreover, the hand-held mainframe 10 is equipped with a display 11, a button set 12, an input end 13, two communication interface ports (not shown), a circuit board having image processing function (not shown), and a memory card slot (not shown); the display 11 is used to display the images captured by the tube module 15 and processed by the circuit board that has image processing function; the button set 12 is used to control the hand-held mainframe 10, such as power, direction control, luminosity control, image capturing and other controlling functions; the input end 13 is used to connect to the flexible cable 14, so that the image data delivered by the flexible cable 14 is input to the hand-held mainframe 10 through the input end 13 to be processed; hence, users can operate through the button set 12 and display the images on the display 11; moreover, the communication interface port (not shown) is to provide the connection between the hand-held mainframe 10 and the outside equipments, such as computer, PDA, monitor, etc to further process the images. The communication interface port can be a USB port; memory card slot (not shown) can provide the hand-held mainframe 10 needed hard disk space for the stored image by inserting all kinds of memory cards. The specification of the memory slot can be CF, MMC, SD, MINI SD, MS, etc.

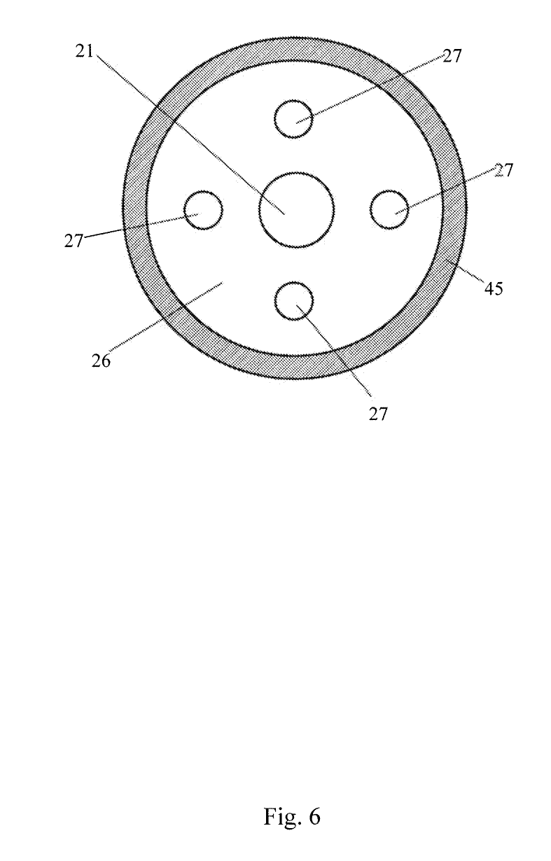

The tube module 15 further includes a probe 20 and a sheath 30, and the first front end 24 of the probe 20 is equipped with a control module 26 (please refer to FIG. 6). On top of the control module 26, an image capturing element 21 having photography and video taping functions and several light sources 27 are further installed and coupled to a plurality of signal wires (not shown). The image capturing element 21 having photography and video taping functions can use a CCD or a CMOS as the sensor accompanied by the optical zooms to capture image data; the light sources 27 are preferably an LED; however, the present invention is not limited to this. These signal wires can be used to deliver the captured image data and send them back to the hand-held mainframe 10 through the flexible cable 14, or used to deliver the control signals from the hand-held mainframe 10 to the control module 26. Those light sources 27 are used when capturing images, and are controlled by the control module 26. When users adjust the button set 12 on the hand-held mainframe 10, the act of the light sources 27 can be controlled, such as a switch, luminosity, etc. The probe 20 can further include a first connection part 22, a handle 25 and an output end 23. The handle 25 is used to be held by users, and the output end 23 is used to be connected to the flexible cable 14, and further connected to the control module 26 using the signal wires, which can be used to transfer image data or control signals; a second front end 35 of the sheath 30 has an opening 31 and a second connection part 32.

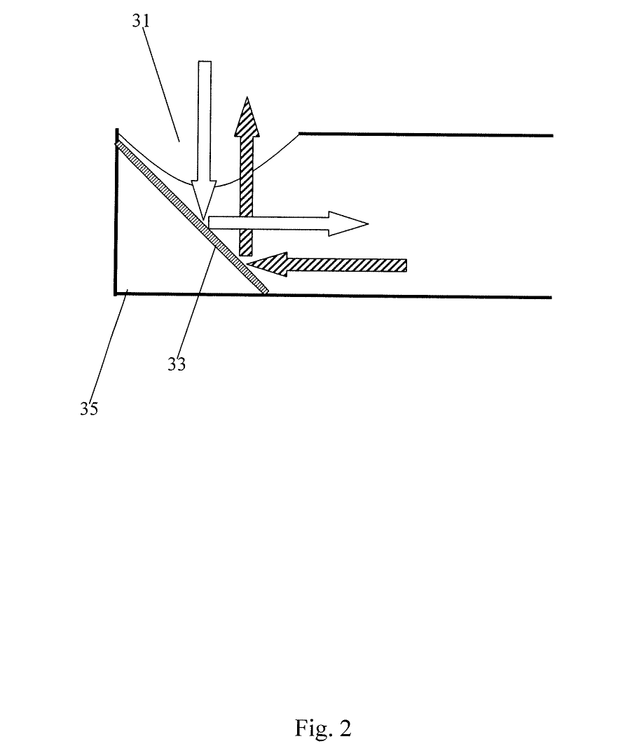

Please refer to FIG. 2, the interior structure of the second front end 35 of the sheath 30 should be able to be understood from which. The shape of the opening 31 can be round, square, etc. However, the present invention is not limited to these shapes, and the inside of the sheath 30 that is corresponding to the opening 31 is equipped with a mirror 33 (as illustrated in the figure). The mirror 33 and the sheath 30 include an angle ranging from 20 to 80 degrees and the preferred angle is 45 degrees. The present invention will be illustrated using 45 degrees below. Through the principle of reflecting light, the direction of the light that can only be shot straight can now be changed into a perpendicular direction to capture the images from different perspectives.

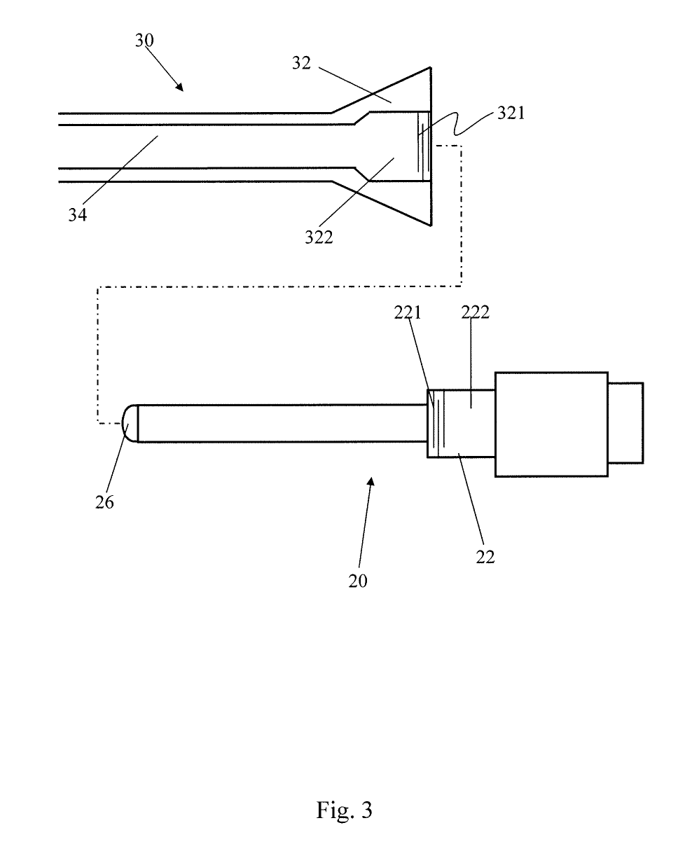

Please refer to FIG. 3, FIG. 3 is the schematic diagram showing the mutual mounting between the sheath and the probe of the present invention. It can be learned from the figure that, the probe 20 and the sheath 30 each has a first connection part 22 and a second connection part 32; Furthermore, the first connection part 22 and the second connection part 32 are the male and female structure that match each other. The passage 34 in the sheath 30 is used to accommodate the probe 20; and the first connection part 22 has a first screw thread area 221 and a first smooth area 222, and the first screw thread area 221 has a plurality of screw thread; the second connection part 32 has a second screw thread area 321 and a second smooth area 322. The second screw thread area 321 has a plurality of screw threads. When the sheath 30 is mounted on the outside of the probe 20, the sheath 30 and the probe 20 can be connected through the screw threads of the first screw thread area 221 and the second screw thread area 321. When the second screw thread area 321 of the first screw thread area 221 is further screwed into the second smooth area 322 (i.e. the screw thread of the second thread area 321 is further screwed to the smooth area through the first screw thread area 221); since the screw threads do not occlude each other any more, the sheath 30 will be rotated without occluding the probe 20; so that users can rotate the sheath 30 without the sheath 30 coming undone from the probe 20. Hence, the direction the opening 31 on the sheath facing can be decided by rotation; that is, the image capturing angle can be rotated 360 degrees, and if the sheath 30 needs to be taken off, the sheath can be taken off by rotating in the counter direction to make the screw threads occlude each other back again, and then the sheath can be taken off.

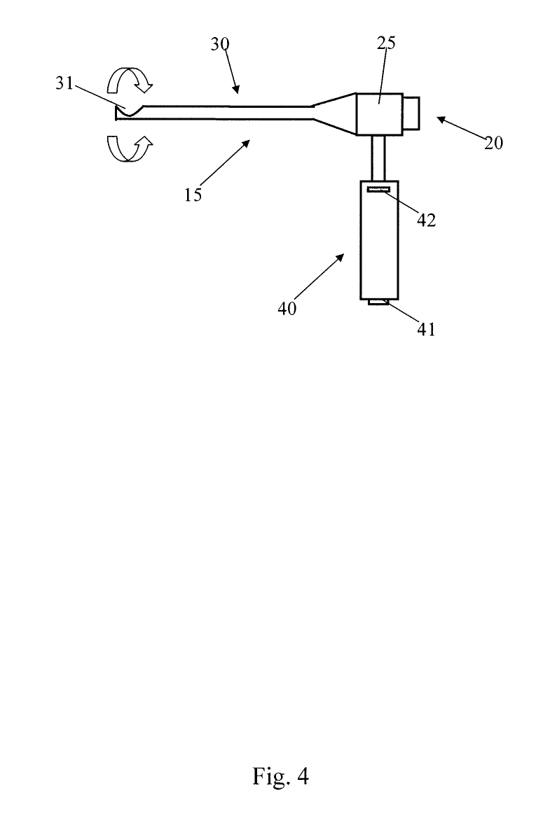

In another embodiment, the handle 25 of the probe 20 of the tube module 15 can be connected to a removable handle 40 on the outside (please refer to FIG. 4). The handle 40 is equipped with a button 41 and a dial 42. The button 41 can control the act of the light source 43 having illuminating function, such as on and off. Moreover, the dial 42 can adjust the luminosity output of the light source 43 by continuous method, for example, the method can be through a gearwheel.

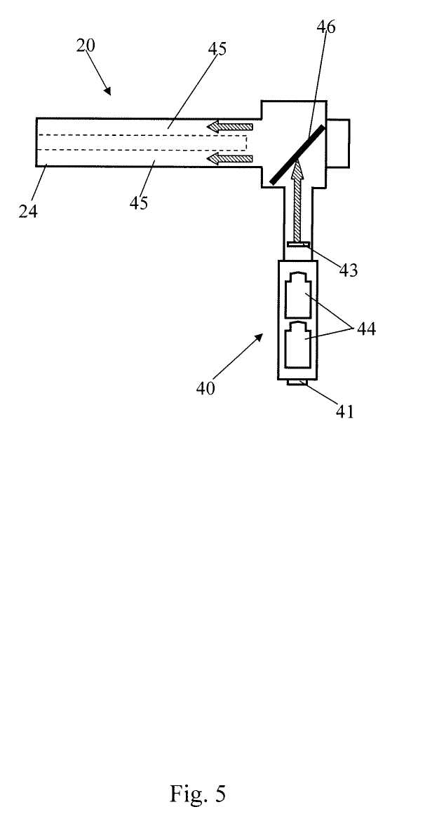

In another embodiment, under the condition that the handle 40 and the probe 20 of the present invention are coupled, the inside of the present invention can further include a lens 46, a light source 43, a plurality of batteries 44 and a light guide passage 45, please refer to FIG. 5, the batteries 44 can be installed in the handle 40 to provide power for the light source 43, so as to generate light; moreover, the on and off of the light source 43 can be controlled by the button 41 on the handle 40, or the luminosity of the light source 43 can be adjusted by the dial 42. In this embodiment, power source of the light source 43 is the batteries 44, but it can also be provided by the hand-held mainframe 10 through the signal wires. The present invention does not limit the source of the power; and the light generated by the light source 43 is focused by the lens 46 and delivered to the light guide passage 45, the light can be transferred to the first front end 24 of the probe 20 through the light guide passage 45 to provide the image capturing element 21 having photography and video taping functions another light source. Please refer to FIG. 5 and FIG. 6, and it should be understood that the light guide passage 45 is located on the outer most surrounding area of the inside of the probe 20. The design of this embodiment can provide extra light source when encountering the problem of not having enough light, or when reaching the probe 20 deep into the space filled with air of low ignition point, the light source 27 included in the first front end 24 of the probe 20 can be turned off in advance and use only the light generated by the light source 43. For example, the operation temperature of an LED ranges from 40 degrees to 60 degrees. If LED is used as the light source and reached down to the space filled with air of low ignition point, the operation temperature might ignite the air and cause explosion or other danger; as a result, this design is designed to meet practical needs and is very popular.

The embodiments above are only used to illustrate the principles and effects of the present invention but not limit the invention. Therefore, a skilled person in the art can modify or change the embodiments mentioned above without departing from the spirit of the present invention. The scope of the present invention is defined by the appended claims listed below.

What is claimed is:

1. A hand-held endoscopic device with a rotation control, including:
a probe, having a first front end with a control module, and the probe is installed with a plurality of signal wires;
a sheath, having a second front end with an opening equipped with a mirror;
a hand-held mainframe; and
a flexible cable, having one end connected to the input end of the hand-held mainframe, and the other end connected to the output end of the probe;
wherein the sheath can be mounted on the outside of the probe and coupled through screw thread, so as to allow users to rotate the direction of the opening of the sheath faces and capture images from different perspectives.

2. The device of claim 1, wherein the control module is further equipped with an image capturing element and a plurality of lighting sources, and delivers the image data or control signals through the signal wires.

3. The device of claim 2, wherein the lighting sources can be a LED.

4. The device of claim 2, wherein the image capturing element can use a CCD or a CMOS to be a sensor and company it with optical lens to capture images.

5. The device of claim 1, wherein the hand-held mainframe is equipped with a display, a button set, an input end, two communication interface, a circuit board having image processing function and a memory card slot.

6. The device of claim 1, wherein the probe has a first connection part, and the connection part has a first screw thread area and a first smooth area.

7. The device of claim 1, wherein the sheath has a second connection part, and the connection part has a second screw thread area and a second smooth area.

8. The device of claim 1, wherein the included angle between the mirror and the sheath ranges from 20 to 80 degrees.

9. A hand-held endoscopic device with a rotation control, including a hand-held mainframe, a flexible cable and a tube module, one end of the flexible cables is coupled to the input end of the hand-held mainframe, and the other end is coupled to the output end of the probe of the tube module, which characterize in that:
a probe, having a control module at its first front end, and a plurality of signal wires are installed in it; and
a sheath, having a second front end has an opening, with a mirror equipped inside;
wherein the sheath can be mounted on the outside of the probe, and coupled through the means of the screw thread, so as to allow users to rotate the direction the opening facing according to the situation, in order to capture images from different perspective.

10. The device of claim 9, wherein the control module is further equipped with a image capturing element and a plurality of lighting sources, and the image data or control signals are delivered through the signal wires.

11. The device of claim 10, wherein the lighting sources can be an LED.

12. The device of claim 10, wherein the image capturing element can use a CCD or a CMOS as a sensor accompanied by optical lens to capture image data.

13. The device of claim 9, wherein the probe has a first connection part, having a first screw thread area and a first smooth area.

14. The device of claim 9, wherein the sheath has a second connection part, having a second screw thread area and a second smooth area.

15. The device of claim 9, wherein the included angle between the mirror and the sheath ranges from 20 to 80 degrees.

16. The device of claim 9, further including a handle equipped with a button and a dial, wherein the button can be used to control the act of the image capturing element, and the dial is used to adjust the direction in which the opening faces through a gearwheel.

17. The device of claim 16, wherein the device further includes a light source which can be turned on or off through the button.

18. The device of claim 17, wherein the illumination strength of the lighting sources included can be adjusted with a dial.

19. The device of claim 17, wherein the power source of the light source can be provided by the signal wires.

20. The device of claim 17, wherein the device can further include a plurality of batteries, and the batteries are to provide the light source power to generate light.

21. The device of claim 17, wherein the device further includes a lens and a light guide passage, and the lens can focus the light generated by the light source and deliver the light source to the light guide passage.
